(19) United States
(12) Patent Application Publication
(10) Pub. No.: US 2005/0096665 A1
(43) Pub. Date: May 5, 2005

(54) Guidewire having a helically contoured portion

(75) Inventors: Brian R. Reynolds, Ramsey, MN (US); James S. Sharrow, Bloomington, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/699,312

(22) Filed: Oct. 30, 2003

(51) Int. Cl. A61F 11/00

(52) U.S. Cl. 606/108

(57) ABSTRACT

A guidewire having a contoured outer surface and methods of making and using the same. The guidewire may include a core wire or member and a jacket disposed over the core member. The jacket may include a contoured outer surface or helically-oriented channel or groove formed therein.

### FIELD OF THE INVENTION

The present invention pertains to medical devices including guidewires. More particularly, the present invention pertains to guidewires with a helically contoured portion that may be disposed near the distal portion of the guidewire, any other suitable portion, or the full length of the guidewire.

### BACKGROUND OF THE INVENTION

A wide variety of devices have been developed for medical use, for example, intravascular use. Some of these devices include guidewires, catheters, and other such devices that each have certain features and characteristics. Among the known medical devices, each has certain advantages and disadvantages. There is an ongoing need to provide alternative designs and methods for making and using medical devices with desirable characteristics and features.

### SUMMARY OF THE INVENTION

The invention provides design, material, and manufacturing method alternatives for medical devices, for example, guidewires. In at least some embodiments, the guidewires include a core wire or member having a proximal region and distal region, a polymer jacket disposed over at least the distal region. The jacket, for example the distal region of the jacket, may include a contoured outer surface including a helically oriented channel or groove. These and some of the other features and characteristics of example embodiments are described in more detail below.

### BRIEF DESCRIPTION OF THE DRAWINGS

FIG. 1 is a partial side view of the distal portion of an example guidewire;

FIG. 1A is a partial side view of the distal portion of another example guidewire;

FIG. 2 is a partial cross-sectional side view of the portion of the guidewire illustrated in FIG. 1;

FIG. 3 is a partial side view of an example guidewire having a coil disposed along a portion of the length thereof;

FIG. 4 is a partial cross-sectional side view of another example distal portion of a guidewire;

FIG. 5 is a partial cross-sectional side view of another example distal portion of a guidewire;

FIG. 6 is a partial cross-sectional side view of another example distal portion of a guidewire;

FIG. 7 is a partial cross-sectional side view of another example guidewire having a coil disposed thereon;

FIG. 8 is a partial cross-sectional side view of the guidewire shown in FIG. 7 after the coil has been removed;

FIG. 9 is an enlarged cross-sectional view of a portion of the guidewire shown in FIG. 8;

FIG. 10 is an enlarged cross-sectional view of a portion of another guidewire;

FIG. 11 is an enlarged cross-sectional view of a portion of another guidewire;

FIG. 12 is a partial cross-sectional side view of another example guidewire; and

FIG. 13 is a partial cross-sectional side view of another example guidewire.

### DETAILED DESCRIPTION OF THE PREFERRED EMBODIMENTS

The following description should be read with reference to the drawings wherein like reference numerals indicate like elements throughout the several views. The detailed description and drawings illustrate example embodiments of the claimed invention.

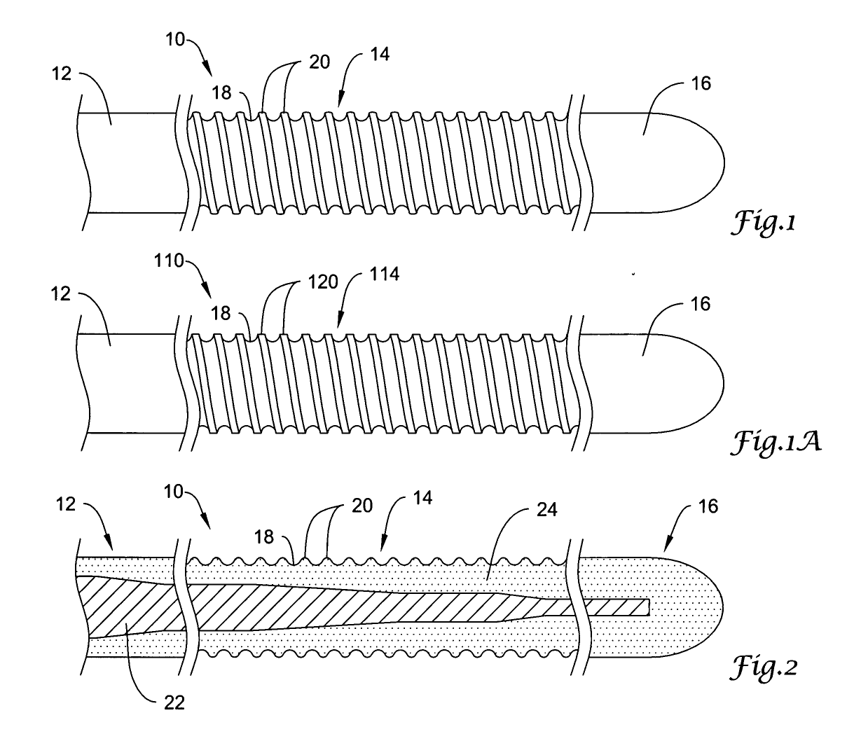

FIG. 1 is a partial cross-sectional side view of a distal portion of an example medical device 10, depicted as a guidewire. Guidewire 10 may include a proximal region 12, a helically contoured or grooved region 14, and a distal region 16. Helically contoured region 14 may provide guidewire 10 with a number of desirable features. The helically contoured region 14 may provide desirable flexibility characteristics. Further, the reduced points of contact within other instruments may reduce friction, for example. When the guidewire is positioned within tissue or a vessel lumen, the contoured surface may assist in maintaining the axial position of the guidewire as tissue fills the contour grooves. As another example, contoured region 14 may comprise a textured surface that is positioned on a portion of the guidewire to improve the ability of a user to grip and/or handle guidewire 10. Moreover, because contoured region 14 is defined by the structure of guidewire 10 and not by a structural element that is added to guidewire 10, it can provide a texture without decreasing the flexibility characteristics of guidewire 10 at contoured region 14. Some of these and other features of guidewire 10 are described in more detail below. Although medical device 10 is depicted in FIG. 1 (and subsequent figures) as a guidewire, the invention is not intended to be limited to guidewires.

Contoured region 14 may have a variety of different configurations. In general, contoured region 14 is defined by a radial groove or channel 18 formed in guidewire 10. This gives contoured region 14 a contoured surface corresponding to channel 18. Channel 18, in preferred embodiments, is arranged in a helical manner about guidewire 10. As described below, the helical arrangement may be the result of forming channel 18 by disposing a coil or tooling wire about guidewire 10 so as to alter the outer surface 20 of guidewire 10, and thereby define channel 18. Additional details regarding the method for manufacturing guidewire 10 and/or forming groove 18 are described in more detail below.

FIG. 2 illustrates guidewire 10 in cross-section in order to show some more of the structural elements that can be included with guidewire 10. Here it can be seen that guidewire 10 may include a core wire or member 22 and a jacket 24 coupled to and/or disposed over core member 22. It can also be seen in FIG. 2 that in at least some embodiments, groove 18 is defined by a groove or channel formed in jacket 24. However, this need not be the case for every contemplated embodiment.

Core member 22 may be made from any suitable material including metals, metal alloys, polymers (including any of those listed herein), or any other suitable material. Some examples of suitable metals and metal alloys include stainless steel, such as 304V, 304L, and 316L stainless steel; alloys including nickel-titanium alloy such as linear elastic or superelastic (i.e., pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 825; or the like; or other suitable materials.

In at least some embodiments, portions or all of core member 22, or other structures included within the guidewire 10 may also be doped with, made of, or otherwise include a radiopaque material. Radiopaque materials are understood to be materials capable of producing a sufficiently bright image on a fluoroscopy screen or another imaging technique during a medical procedure. This image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, core member 22 and/or guidewire 10 may include one or more marker bands or coils that include a radiopaque material.

In some embodiments, a degree of MRI compatibility can be imparted into guidewire 10. For example, to enhance compatibility with Magnetic Resonance Imaging (MRI) machines, it may be desirable to make core member 22, or other portions of guidewire 10, in a manner that would impart a degree of MRI compatibility. For example, core member 22, or portions thereof, may be made of a material that does not substantially distort the image and create substantial artifacts (artifacts are gaps in the image). Certain ferromagnetic materials, for example, may not be suitable because they may create artifacts in an MRI image. Core member 22, or portions thereof, may also be made from a material that the MRI machine can image. Some materials that exhibit these characteristics include, for example, tungsten, Elgiloy, MP35N, nitinol, and the like, and others.

In some embodiments, core member 22 may include multiple pieces or portions. The individual pieces may be made from the same or different materials and may be joined together in any suitable manner. For example, the material used to construct the proximal portion of core member 22 can be relatively stiff for pushability and torqueability, and the material used to construct the distal portion of core member 22 can be relatively flexible by comparison for better lateral trackability and steerability. More particularly, the proximal portion of core member 22 can be formed of straightened 304v stainless steel wire or ribbon, and the distal portion of core member 22 can be formed of a straightened super elastic or linear elastic alloy, for example a nickel-titanium alloy wire or ribbon.

Core member 22 can have a solid cross-section, but in some embodiments, can have a hollow cross-section. In yet other embodiments, core member 22 can include combinations of areas having solid cross-sections and hollow cross sections. Moreover, core member 22 can be made of rounded wire, flattened ribbon, or other such structures having various cross-sectional geometries. The cross-sectional geometries along the length of core member 22 can also be constant or can vary. For example, FIG. 2 depicts core member 22 as having a round cross-sectional shape. It can be appreciated that other cross-sectional shapes or combinations of shapes may be utilized without departing from the spirit of the invention. For example, the cross-sectional shape of core member 22 may be oval, rectangular, square, polygonal, and the like, or any suitable shape.

As shown in FIG. 2, a portion of core member 22 (generally disposed adjacent the contoured region 14 and distal region 16 of guidewire 10) may include one or more tapers or tapered regions. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. Although FIG. 2 depicts the distal portion of core member 22 as being tapered, it can be appreciated that essentially any portion of core member 22 and/or guidewire 10 may be tapered and the taper can be in either the proximal or the distal direction. As shown in FIG. 2, the tapered region may include one or more portions where the outside diameter is narrowing, for example, the tapered portions, and portions where the outside diameter remains essentially constant, for example, constant diameter portions. The number, arrangement, size, and length of the narrowing and constant diameter portions can be varied to achieve the desired characteristics, such as flexibility and torque transmission characteristics. The narrowing and constant diameter portions as shown in FIG. 2 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing the core wire during the grinding process. In some embodiments, core member 22 can be centerless ground using a Royal Master HI-AC centerless grinder.

In some embodiments, distal region 16 defines the distal tip of guidewire 10. However, this need not be the case as a number of other types of guidewire tips are contemplated. For example, in addition to being a generally atraumatic tip, the distal tip of guidewire 10 may include a spring-type tip, a solder ball tip, a polymer ball tip, and the like, or any other suitable tip.

The process of defining groove 18 may include disposing and/or embedding a tooling wire 26 over or into contoured region 14 (i.e., over or into the portion of guidewire 10 that will include groove 18 and thereby define contoured region 14) and then removing the tooling wire as shown in FIG. 3. The embedding process (which may be described as thermal embedding or tension embedding) may vary, but generally includes disposing tooling wire 26 over jacket 24 and heating. In at least some embodiments, tooling wire 26 is wound under tension about jacket 24. Heating may occur at a number of different temperatures. For example, heating may occur at about 30° C. to about 300° C. or more. The depth that tooling wire 26 embeds within jacket 24 may be affected by the amount of tension and the hardness of jacket 24 (or any other structure that tooling wire 26 may be wound about). For example, as more tension is applied tooling wire 26 may embed deeper within jacket 24. Similarly, tooling wire 26 will embed deeper into guidewires that utilize jacket 24 that is made from a softer material.

Alternatively, the coiling tension may allow tooling wire 26 to recover in wound diameter (i.e., “shrink” to the diameter that tooling wire 26 would have if the tension was relieved) when jacket 24 is heated. Therefore, the diameter of tooling wire 26 reduces as heat is applied (i.e., the tension within tooling wire 26 is relieved) and tooling wire 26 moves inward into jacket 24 as the outer surface of jacket 24 wicks and/or otherwise changes shape to conform to the inside surface of tooling wire 26 (or take on some other shape). Thus, the shifting of tooling wire 26 and the alteration of jacket 24 results in the embedding of tooling wire 26 within jacket 24. In still other alternative embodiments, the jacket 24 can be heated prior to wrapping the coil, such that the coil at least partially embeds during winding. Further heating may be required to achieve the desired surface profile.

Being “embedded” within jacket 24 is understood to mean being disposed over jacket 24 in a manner that alters the shape of the outer surface 20 of jacket 24. Thus, tooling wire 26 is implanted or entrenched within jacket 24 and is not simply disposed on the top of jacket 24, completely submerged within jacket 24, or disposed between jacket 24 and another layer of material. Jacket 24 (in the absence of tooling wire 26) may have or be manufactured to have a smooth outer surface. Embedding tooling wire 26 into jacket 24 changes the shape of the outer surface as tooling wire 26 is embedded therein. For example, embedding tooling wire 26 into jacket 24 may result in jacket 24 wicking between the individual windings of tooling wire 26. Accordingly, the shape of the outer surface 20 of jacket 24 may be wave-like or otherwise include a series of peaks or alternating peaks and valleys. In some embodiments, these peaks in outer surface 20 are generally rounded. This may be because outer surface 20 tends to curve toward tooling wire 26 in areas adjacent where tooling wire 26 is wound about and contacts jacket 24. In addition, heating may also tend to round the peaks formed in outer surface 20. However, this need not be the case. For example, FIG. 1A depicts guidewire 110 where the peaks in outer surface 120 of contoured region 114 are essentially unaltered and appear squared or flattened.

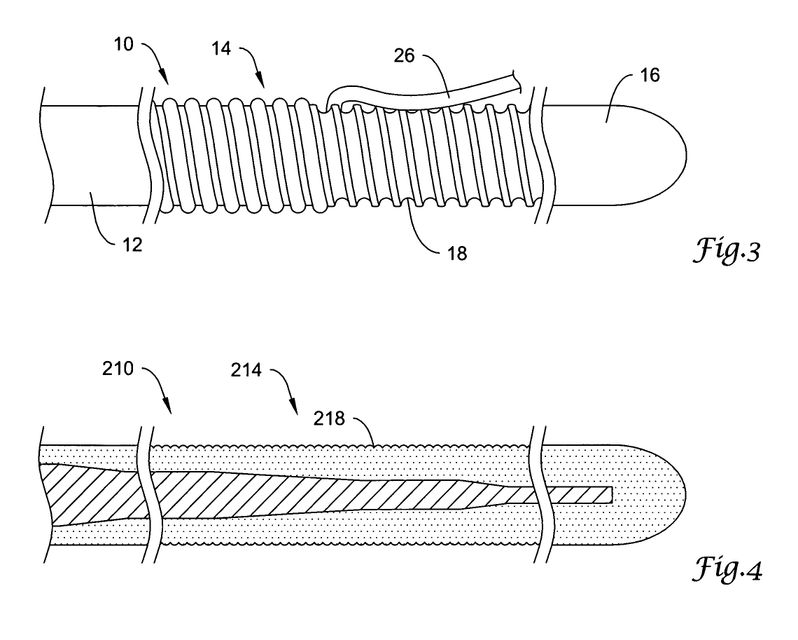

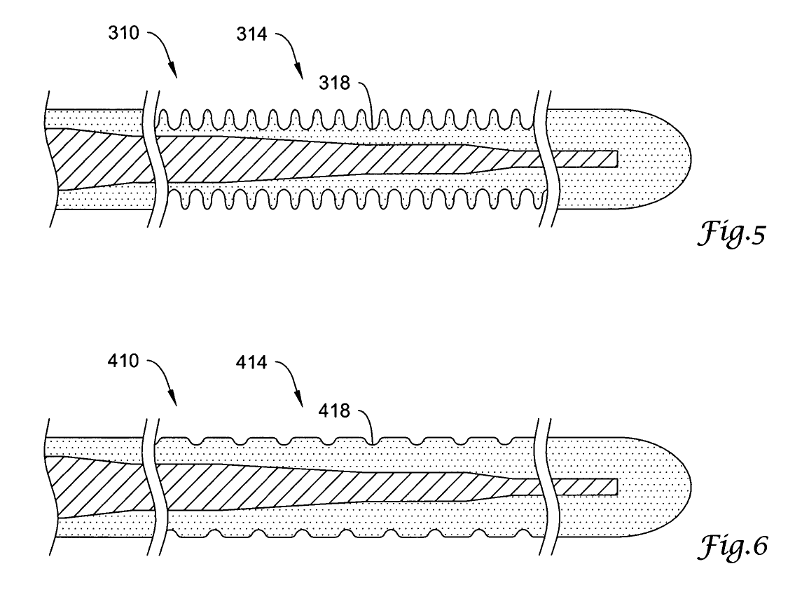

Contoured region 14 could also have a number of different shapes or contours. Some of these shapes may be the result of altering the thickness of tooling wire 26 or altering the depth that tooling wire 26 is embedded into jacket 24. Some examples of alternative shapes or profiles for contoured regions are shown in FIGS. 4-6. For example, FIG. 4 depicts guidewire 210 where contoured region 214 is defined by a relatively small or shallow groove 218 or sets of grooves 218. Groove 218 may be described as being crowned and/or tightly packed and may be formed by utilizing a tooling wire 26 wound in a relatively tight or closed pitch. Making this embodiment may include utilizing a smaller or thinner tooling wire to define groove 318. In contrast, FIG. 5 depicts guidewire 310 where contoured region 314 is defined by a larger or deeper groove 318 or set of grooves 318. Grooves 318 may be described as being loosely packed or formed from a tooling wire 26 wound in a relatively loose or open pitch. Making this embodiment may include utilizing a larger or thicker tooling wire to define groove 318. Similarly, FIG. 6 depicts guidewire 410 where contoured region 414 is defined by one or more grooves 418 that define one or more flattened peaks radiused or rounded on each side. It can be appreciated that a number of alternative contoured regions are contemplated that include a variable-depth groove (i.e., the groove is embedded deeper in some parts of the contoured regions than others), combinations of deep and narrow grooves, or any other suitable arrangement.

The materials used for tooling wire 26 and jacket 24 can vary greatly and may include any suitable material. It may be desirable, however, for the materials to be chosen based upon their ability to facilitate the embedding process and so as to achieve the desired level of embedding. For example, jacket 24 may be made from a thermoplastic material (i.e., a material whose viscosity changes with the induction of heat), a thermoplastic-like material, a thermoset material, combinations thereof, or the like. Some examples of these types of materials are listed below. Tooling wire 26 can be made from fluorocarbon polymer or include a central core material 22 with a fluorocarbon coating 24. These materials may be desirable because of the ability of the thermoplastic material to “flow” or otherwise change shape when heated. Thus, tooling wire 26 can be disposed adjacent the thermoplastic jacket 24 so that when heat is applied, the viscosity of jacket 24 changes and/or softens or flows, which facilitates the embedding of tooling wire 26 within jacket 24.

When these materials are used, tooling wire 26 is embedded within jacket 24 without melding together the two structures. Thus, a thermal bond is not defined that attaches tooling wire 26 with jacket 24 along the region where tooling wire 26 is embedded. This feature may be desirable, because creating a direct bond between tooling wire 26 and jacket 24 could create a position where the flexibility and/or bending characteristics of guidewire 10 are altered. This may create regions of inflexibility along guidewire 10, which may be undesirable. Moreover, selecting these materials may enhance the ability of a technician to remove tooling wire 26 from jacket 24 (which is illustrated in FIG. 3 where a portion of tooling wire 26 has been removed exposing groove 18).

As stated above, defining contoured region 14 may provide guidewire 10 with a number of desirable features. For example, by defining contoured region 14 as a radially-inward groove in jacket 24, the overall profile of guidewire 10 can be kept relatively small. Thus, at least a portion of the extra outside diameter or profile that may have been added by disposing tooling wire 26 onto jacket 24 can be eliminated. Accordingly, guidewire 10 can be easily sized for sensitive areas such as the central nervous system (where guidewire 10 may have an outside diameter of about 0.012 inches or less), for interventions near the heart (where guidewire 10 may have an outside diameter in the range of about 0.010 to about 0.018 inches or so), and for peripheral interventions (where guidewire 10 may have an outside diameter of about 0.014 inches to about 0.050 inches or more). Additionally, contoured region 14 defines a helical or otherwise textured outer surface along a portion of the length of guidewire 10. This textured outer surface may improve traction between guidewire 10 and another device, such as a catheter. For example, guidewire 10 may be used in conjunction with a number of different intravascular interventions where a catheter or other device is advanced over guidewire 10. At some point during the intervention, it may be desirable to maintain the position of guidewire 10 relative to the catheter. Because guidewires may be highly lubricous, maintaining their position within the catheter could pose a challenge. Accordingly, defining a textured surface on the outside of guidewire 10 may help improve the traction (e.g., by ratcheting or catching on the catheter lumen or catheter tip) between guidewire 10 and the catheter lumen while adding or maintaining lubricity (e.g., by reducing the surface area touching the catheter lumen, thereby reducing friction). Additionally, the textured surface may also improve the traction between guidewire 10 and the tissue that it may interact with. For example, endothelial cells or other vessel tissue may grip or otherwise hold onto the textured surface and thereby improve traction. Finally, grooved region 16 may define a textured surface that improves the ability of a user to grasp and hold onto guidewire 10.

Although it is stated above that jacket 24 may be made from a thermoplastic, any suitable polymer made be used. Some examples of suitable polymers (including thermoplastics) may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, polyethylene (PE), Marlex high-density polyethylene, Marlex low-density polyethylene, linear low density polyethylene (for example, REXELL®), polyethylene terephthalate (PET), polyetheretherketone (PEEK), polyimide (PI), polyetherimide (PEI), polyphenylene sulfide (PPS), polyphenylene oxide (PPO), polysulfone, nylon, perfluoro(propyl vinyl ether) (PFA), low durometer thermal plastics (e.g., 25-50 Sure D), tungsten loaded thermal plastic compound, bismuth subcarbonate loaded thermal plastic compound, barium sulfate loaded thermal plastic compound, other suitable materials, or mixtures, combinations, copolymers thereof, polymer/metal composites, and the like. In some embodiments, jacket 24 can be blended with a liquid crystal polymer (LCP). For example, the mixture can contain up to about 5% LCP.

The tooling wire 26 used to define contoured region 14 may be made from a solid fluorocarbon material such as PTFE or otherwise include outer coating 24 that is made from a fluorocarbon. A number of other materials may be used. For example, tooling wire 26 may be made from a molecularly oriented high modulus and high melt index thermal plastic, a polymer clad tungsten or stainless steel wire (that is unlikely to thermally recover with heat), and the like, or any other suitable material including any of those listed herein. Tooling wire 26 may also vary in size, length, shape, pitch, and the like. For example, tooling wire 26 can have a generally round cross-sectional shape, a flattened ribbon-like shape, or any other suitable shape. Moreover, the pitch may be constant or vary, and can include tightly pitched regions, loosely pitched regions, and combinations thereof. It can be appreciated that as the size, length, shape, pitch, or other properties of tooling wire 26 change, the resultant groove 18 formed by tooling wire 26 analogously changes. For example, if tooling wire 26 has a squared shape, the groove 18 defined by tooling wire 26 would similarly have a squared shape.

The lengths of regions 12/14/16 of guidewire 10 are typically dictated by the length and flexibility characteristics desired in the final medical device. For example, proximal region 12 may have a length in the range of about 20 to about 300 centimeters or more, contoured region 14 may have a length in the range of about 3 to about 50 centimeters or so, and distal region 16 may have a length in the range of about 3 to about 50 centimeters or more. It can be appreciated that alterations in the length of regions 12/14/16 can be made without departing from the spirit of the invention.

Tooling wire 26 may be used to define contoured region 14 at essentially any position along the length of guidewire 10. For example, tooling wire 26 may be used to define contoured region 14 between proximal region 12 and distal region 16. Therefore, contoured region 14 may be set back proximally (e.g., about 3 to 50 centimeters, depending on the length of distal region 16) from the distal end of guidewire 10. According to this embodiment, distal region 16 (i.e., the portion of guidewire 10 that is disposed distally of contoured region 14) has a generally smooth outer surface and defines a smooth distal tip. This smooth distal tip may desirably impact the crossing ability of guidewire 10. This arrangement, however, is not intended to be limiting because other arrangements are contemplated. For example, contoured region 14 could extend to the proximal end, distal end, or along the entire length of guidewire. Alternatively, guidewire 10 may include multiple contoured regions 18 that are intermixed with regions that are not contoured and dispersed along any portion of guidewire 10.

In some embodiments, contoured region 14 may be subjected to an additional heating step after tooling wire 26 is removed. This heating may result in contoured region 14 having a somewhat more rounded shape or configuration as shown in FIG. 4. This heating step may comprise essentially any suitable heating method including, but not limited to, infrared reflow heating. Additionally, this heating step could occur at any essentially any suitable point in the manufacturing process or not be included at all.

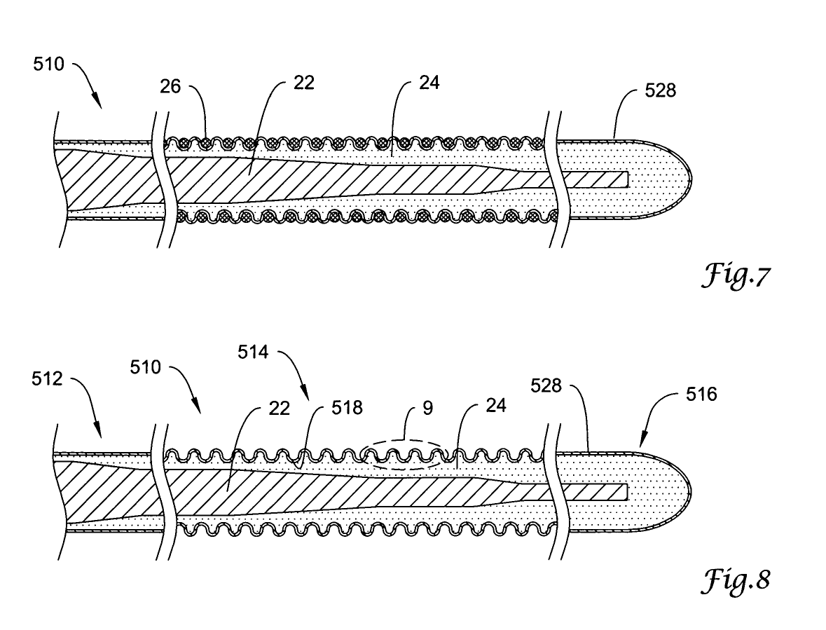

FIG. 7 illustrates guidewire 510, similar to any of the other guidewires described herein, that includes a coating 528. Coating 528 may comprise a lubricious, a hydrophilic, a protective, or other type of coating that may provide guidewire 510 with a number of desirable features. For example, hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Coating 528 may be formed, for example, by coating, extrusion, co-extrusion, interrupted layer co-extrusion (ILC), or fusing several segments end-to-end over guidewire 510. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

It can be seen in FIG. 7 and FIG. 8 that tooling wire 26 may be disposed over and/or embedded within coating 528 and jacket 24 (FIG. 7) and then removed (FIG. 8) so that contoured region 514 is defined by a number of grooves 518 in coating 528 and jacket 24. As in guidewire 10, contoured region 514 may be positioned between proximal region 512 and distal region 516 of guidewire 510 or at any other suitable position.

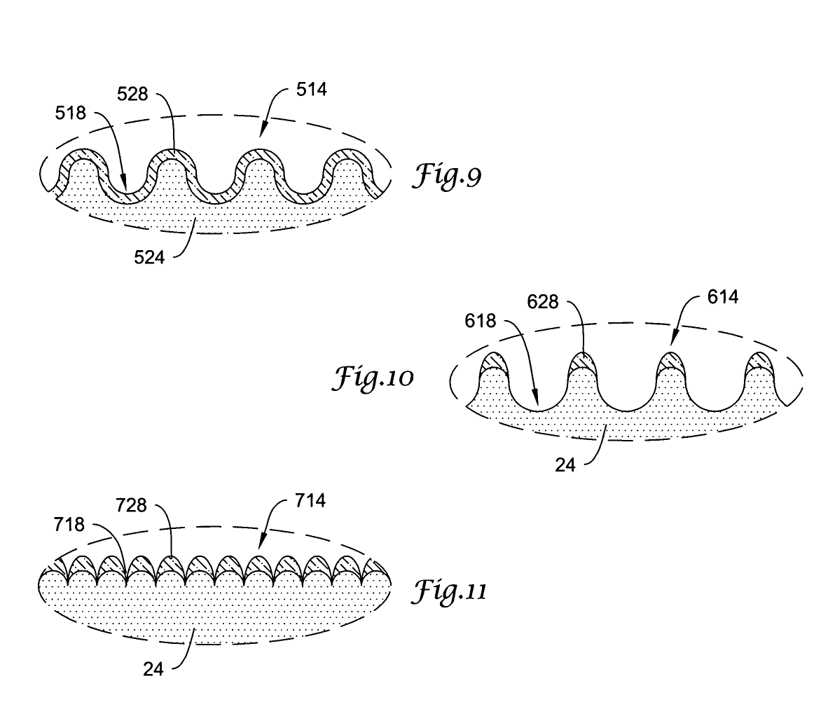

When tooling wire 26 is wound about guidewire 510 (or any other guidewire described herein) after coating 528 has been disposed on jacket 24, the result may be that coating 528 essentially follows or traces the path of jacket 24 and/or groove 518 as shown in FIG. 9. In some other embodiments, however, this may not be the case. For example, FIG. 10 illustrates and enlarged view of contoured region 614 where coating 628 does not trace groove 618. Instead, coating 628 tends to remain on the outer surface of jacket 24 (i.e., away from groove 618) so that the outer surface of contoured region 614 includes the lubricious coating 628 while groove 618 is generally free of coating 618. This arrangement may be desirable, for example, by allowing the outer surface of to remain sufficiently slippery or lubricious while reducing the potential impact of coating 628 on the flexibility of the guidewire. FIG. 11 depicts a similar effect for contoured region 714 where coating 728 tends to remain on the outer surface of jacket 24 (in an embodiment similar to guidewire 210 shown in FIG. 4, but with coating 728) and groove 718 is substantially free of coating 728.

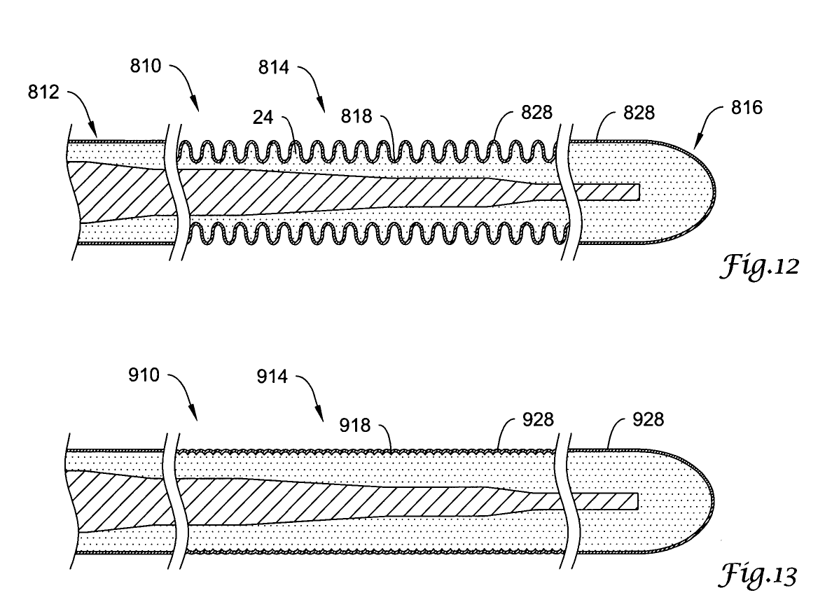

FIGS. 12-13 depict other example guidewires where the coating is applied after the contoured region is defined in jacket 24. For example, FIG. 12 depicts guidewire 810 where contoured region 814 (disposed between proximal region 812 and distal region 816) is coated by coating 828 after contoured region 814 is formed in jacket 24. Coating 828 is generally similar to the other coatings described above. In some embodiments, coating 828 may follow the contour of contoured region 814, thereby tracing the contour defined by groove 818. However, this need not be the case, as portions or all of coating 828 may “fill” groove 818 and provide a smooth outer surface for portions of contoured region 814. Similarly, FIG. 13 depicts guidewire 910 where coating 928 is applied after contoured region 914 is defined in jacket 24. Guidewire 910 is essentially the same as guidewire 810 except that groove 914 is smaller or shallower than groove 814.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

1. A medical device, comprising:
an elongate core member;
a polymer jacket disposed over at least a portion of the core member, the polymer jacket having a textured outer surface; and
wherein the textured outer surface is defined by a helical groove formed in the outer surface of the polymer jacket.

2. The medical device of claim 1, further comprising a coating disposed over the polymer jacket.

3. The medical device of claim 1, wherein the helical groove has rounded edges.

4. The medical device of claim 1, wherein the polymer jacket is free of a coil.

5. The medical device of claim 1, wherein the helical groove is positioned proximally of a distal tip of the medical device.

6. An intravascular guidewire, comprising:
an elongate core member having a proximal end region and a distal end region;
a polymer jacket disposed over the distal end region of the core member, the polymer jacket having a textured outer surface; and
wherein the textured outer surface is defined by a helical channel formed in the outer surface of the polymer jacket.

7. The guidewire of claim 6, further comprising a coating disposed over the polymer jacket.

8. The guidewire of claim 6, wherein the helical channel has rounded edges.

9. The guidewire of claim 6, wherein the polymer jacket is free of a coil.

10. The guidewire of claim 6, wherein the textured outer surface of the polymer jacket is positioned proximally a distance from a distal end of the polymer jacket.

11. The guidewire of claim 10, wherein the textured outer surfaced is positioned about 2 to about 20 centimeters from the distal end of the polymer jacket.

12. A method for manufacturing a medical device, comprising the steps of:
providing a core member having a proximal region and a distal region;
disposing a polymer jacket over the distal region of the core member, the polymer jacket having an outer surface;
winding a coil over the polymer jacket;
heating the polymer jacket so that the coil alters the outer surface of the polymer jacket; and
removing the coil from the polymer jacket.

13. The method of claim 12, wherein the step of winding a coil over the polymer jacket includes winding the coil under tension over the polymer jacket.

14. The method of claim 12, further comprising the step of disposing a coating over the polymer jacket.

15. The method of claim 14, wherein the step of disposing a coating over the polymer jacket precedes the step of winding a coil over the polymer jacket.

16. The method of claim 14, wherein the step of disposing a coating over the polymer jacket follows the step of winding a coil over the polymer jacket.

17. The method of claim 16, wherein the step of disposing a coating over the polymer jacket follows the step of removing the coil from the polymer jacket.

18. The method of claim 17, wherein the step of disposing a coating over the polymer jacket includes disposing the coating over the altered surface of the polymer jacket.

19. The method of claim 18, wherein the polymer jacket follows the altered surface of the polymer jacket.

20. A method for manufacturing a medical device, comprising the steps of:
providing a core member;
disposing a polymer jacket over the core member, the polymer jacket having an outer surface;
winding a coil over the polymer jacket, wherein the coil is wound under tension;
heating the polymer jacket so that the coil tension is relieved and the coil embeds within the outer surface of the polymer jacket; and
removing the coil from the jacket, thereby leaving a helically-contoured groove corresponding to where the coil was embedded in the polymer jacket.

21. The method of claim 20, further comprising the step of disposing a coating over the polymer jacket.

22. The method of claim 21, wherein the step of disposing a coating over the polymer jacket precedes the step of winding a coil over the polymer jacket.

23. The method of claim 21, wherein the step of disposing a coating over the polymer jacket follows the step of winding a coil over the polymer jacket.

24. The method of claim 23, wherein the step of disposing a coating over the polymer jacket follows the step of removing the coil from the polymer jacket.

25. The method of claim 24, wherein the step of disposing a coating over the polymer jacket includes disposing the coating over the altered surface of the polymer jacket.

26. The method of claim 25, wherein the polymer jacket follows the altered surface of the polymer jacket.

27. The method of claim 20, further comprising a second heating step.

28. The method of claim 27, wherein the second heating step occurs after the step of removing the coil from the polymer jacket.

29. The method of claim 27, wherein the second heating step smoothes the helically-contoured groove.

30. A method for manufacturing a medical device, comprising the steps of:
providing a core member;
disposing a polymer jacket over the core member, the polymer jacket having an outer surface;
winding a coil over the polymer jacket, wherein the coil is wound under tension;
embedding the coil within the outer surface of the polymer jacket; and
removing the coil from the jacket, thereby leaving a helically-contoured groove in the polymer jacket at a position corresponding to where the coil was embedded in the polymer jacket.

31. The method of claim 30, further comprising the step of heating the helically-contoured groove.

32. The method of claim 30, wherein the step of embedding the coil within the outer surface of the polymer jacket includes heating.
